(19) United States
(12) Patent Application Publication
(10) Pub. No.: US 2021/0283359 A1
(43) Pub. Date: Sep. 16, 2021

(54) CONTINUOUS POSITIVE AIRWAY PRESSURE (CPAP) DEVICES AND SYSTEMS

(71) Applicant: University of Tennessee Research Foundation, Knoxville, TN (US)

(72) Inventors: Maddie Singer, Memphis, TN (US); Kristen Archbold, Germantown (US); Timothy L. Hottel, Memphis, TN (US); Russell A. Wicks, Memphis, TN (US)

(73) Assignees: University of Tennessee Research Foundation, Knoxville (US); University of Tennessee Research Foundation, Knoxville (US)

(21) Appl. No.: 16/338,899

(22) PCT Filed: Oct. 3, 2017
(86) PCT No.: PCT/US2017/054936

§ 371 Date: Apr. 2, 2019

Related U.S. Application Data
(60) Provisional application No. 62/403,359, filed on Oct. 3, 2016.

(51) Int. Cl. A61M 16/06; A61M 16/08; A61M 16/22

(52) U.S. Cl. CPC A61M 16/0683; A61M 16/0666; A61M 2205/3334; A61M 16/22; A61M 16/0816

(57) ABSTRACT

Continuous positive airway pressure (CPAP) devices and systems including patient-specific mandibular and nasal component, configured to deliver pressurized air to nasal and oral passageways of a subject. Mask-less and strap-less CPAP devices designed to provide dual delivery of continuous pressurized air to a mouth and nose of a subject. Methods of treating and ameliorating sleep apnea, hypopnea syndrome, and obstructive sleep apnea.

### CROSS-REFERENCE TO RELATED APPLICATIONS

This application claims benefit of U.S. Provisional Patent Application Ser. No. 62/403,359, filed Oct. 3, 2016, herein incorporated by reference in its entirety.

### TECHNICAL FIELD

The presently disclosed subject matter is directed to continuous positive airway pressure (CPAP) devices and systems. More particularly, the presently disclosed subject matter is directed to devices and systems configured to deliver pressurized air to an oral and/or nasal passageway of a subject to treat sleep apnea and related conditions.

### BACKGROUND

Sleep apnea, or hypopnea syndrome or obstructive sleep apnea (OSA), is defined as intermittent and repetitive episodes of breathing cessation that restrict the amount of oxygen intake. According to the World Health Organization (WHO), about one in fifteen adults worldwide suffer from moderate or severe sleep apnea, yet only a fraction of individuals have been diagnosed. The market is not declining and the trend is rapidly expanding in size and diversity.

A common therapy for sleep apnea and related conditions is the use of a continuous positive airway pressure (CPAP) unit. A CPAP unit or system is a machine that delivers lightly pressurized air through a hose that is hooked to a mask that a subject wears while sleeping. The flow of air from CPAP unit acts like a splint to keep the subject's upper airway open to thereby reduce and/or prevent sleep apnea.

Often times sleep apnea patients do not wear their CPAP masks at night since the mask, cords, hoses, etc. can be uncomfortable while trying to sleep. For example, some studies show that only 23%-45% of patients for whom CPAP is prescribed actually use the machine on a regular basis. Additionally, CPAP users average only 4-5 hours of sleep per night versus the 7-9 hours of recuperative sleep recommended by organizations such as the National Sleep Foundation.

Additionally, CPAP intolerance frequently cited by patients include concerns about the mask leaking and/or an inability to get a particular design to fit properly; claustrophobia; uncomfortable straps and headgear; latex allergies; noise from the CPAP machine that disturbs sleep of the patient (or his/her bed partner); restricted movement during sleep because of the CPAP apparatus; nasal and/or breathing discomfort (dryness, congestion); and/or general physical discomfort (condensation build-up, fit, weight) in the case of masks. These and other concerns can in some embodiments be addressed by the devices and systems of the instant disclosure. For example, with the presently disclosed devices and systems there is no issue with mask leaking as the devices are custom-made for the patient, and there are no uncomfortable straps.

For some patients, including those with mild-to-moderate OSA, an oral sleep appliance may eliminate the need for a CPAP device or surgery. For patients with more severe sleep-disordered breathing problems, an oral sleep appliance may be a convenient adjunct therapy. This recommendation is consistent with the American Academy of Sleep Medicine's practice parameters. The presently disclosed devices and systems can in some embodiments be considered a medical appliance that is still a machine-linked CPAP, versus an oral appliance (OA).

Moreover, there is growing evidence to support the importance of effectively diagnosing and treating OSA in children, especially considering it is now known that OSA can have lasting/long-term effects on child development, learning and behavior. If the diagnostic can confer a diagnosis quickly and in a cost effective manner, the field could greatly benefit from the technology. The disclosed devices and systems can in some embodiments address this growing need.

Conventionally, within the therapeutic device segment, there are generally two non-invasive product areas based on applying positive airway pressure (PAP): namely flow generators and facial interfaces. One invasive category of surgically implantable neurostimulators completes this segment. For the non-invasive products, flow generator technology is further divided into: continuous positive airway pressure, or CPAP, Bi-Level PAP (Positive Airway Pressure), and Auto-PAP devices. The facial interfaces area usually refers to dental or other OA therapy. With OA therapies, several parameters must be considered such as the health of the dentition, the presence of potentially affecting parasomnias, a reliable and clear polysomnogram, evaluations of bony and soft tissue anatomy, and health, among others.

However, there is still an unment need for effective CPAP devices and systems that effectively treat sleep apnea and related conditions, and provide a user with a comfortable user experience that improves compliance.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are continuous positive airway pressure (CPAP) devices and/or systems comprising a mandibular component, a nasal component, a main body connected to and in fluid communication with the mandibular component and the nasal component, and a hose coupler configured to receive a hose providing pressurized air from an air source, wherein the main body is configured to deliver the pressurized air from the air hose to one or both of the mandibular component and the nasal component, wherein the mandibular component is configured to deliver pressurized air to an oral passageway of a subject, wherein the nasal component is configured to deliver pressurized air to a nasal passageway of a subject. In some aspects, the mandibular component comprises an impression of a subject's teeth. In some aspects, the mandibular component comprises an upper portion and lower portion configured to conform to the upper and lower teeth, respectively, of a subject. In some aspects, the devices and/or systems can be configured to be retained on a subject's face by oral retention on the mandibular component. In some aspects, the mandibular component comprises an oral air passage configured to deliver pressurized air to an oral passageway of a subject.

In some aspects, the nasal component comprises an impression of a subject's nose. In some aspects, the nasal component comprises one or more cannulae configured to position the nasal component on a subject's nose, and configured to deliver pressurized air from the main body to the subject's nose. In some aspects, the nasal component further comprises two nasal air passages configured to align with a subject's nostrils.

In some aspects, the mandibular component is configured to be positioned in a subject's mouth to create a substantially air-tight seal for the oral passageway, wherein the nasal component is configured to be positioned on a subject's nose to create a substantially air-tight seal for the nasal passageway. In some aspects, the devices and/or systems can be configured to provide dual delivery of pressurized air to both the nose and mouth. In some aspects, the device and/or system is devoid of retaining straps and a mask.

In some aspects, the devices and/or systems can further comprise a CO₂ exchanger configured to allow expired air to exit the device and/or system. In some aspects, the device and/or system further comprises an air pump and hose together configured to deliver lightly pressurized air to the device and/or system. The devices and/or systems can be configured to attach to an existing air pump and hose. In some embodiments, the mandibular component and/or nasal component can be removable and/or replaceable.

In some aspects, the disclosed CPAP devices and/or systems can be configured to deliver pressurized air to a subject's airway to keep the subject's upper airway open to thereby reduce and/or prevent sleep apnea. In some aspects, the devices and/or systems can be configured to be used with an adult subject and/or an adolescent subject.

Provided herein are also mask-less and/or strap-less CPAP devices configured to provide dual delivery of continuous pressurized air to a mouth and nose of a subject, the device comprising a patient specific mandibular component and patient specific nasal component, wherein the device is configured to be retained in place by oral retention on the mandibular component. In some aspects, the mask-less and/or strap-less CPAP devices can comprise a main body connected to and in fluid communication with the patient specific mandibular component and the patient specific nasal component, and a hose coupler configured to receive a hose providing pressurized air from an air source, wherein the main body is configured to deliver the pressurized air from the air hose to one or both of the patient specific mandibular component and the patient specific nasal component, wherein the patient specific mandibular component is configured to deliver pressurized air to an oral passageway of a subject, wherein the patient specific nasal component is configured to deliver pressurized air to a nasal passageway of a subject.

In some aspects, the devices can be configured to be used by a subject that is a mouth breather, nose breather, or both. In some aspects, the patient specific mandibular component can comprise an impression of a subject's teeth. In some aspects, the patient specific mandibular component can comprise an upper portion and lower portion configured to conform to the upper and lower teeth, respectively, of a subject. In some aspects, the device can be configured to be retained on a subject's face by oral retention on the patient specific mandibular component. In some aspects, the patient specific mandibular component can comprise an oral air passage configured to deliver pressurized air to an oral passageway of a subject.

In some aspects, the patient specific nasal component can comprise an impression of a subject's nose. In some aspects, the patient specific nasal component can comprise one or more cannulae configured to position the patient specific nasal component on a subject's nose, and configured to deliver pressurized air from the main body to the subject's nose. In some aspects, the patient specific nasal component can further comprise two nasal air passages configured to align with a subject's nostrils.

In some aspects, the patient specific mandibular component can be configured to be positioned in a subject's mouth to create a substantially air-tight seal for the oral passageway, wherein the patient specific nasal component is configured to be positioned on a subject's nose to create a substantially air-tight seal for the nasal passageway. In some aspects, the device can be configured to provide dual delivery of pressurized air to both the nose and mouth.

In some aspects, the device can be devoid of retaining straps and a mask. In some aspects, the device can further comprise a CO₂ exchanger configured to allow expired air to exit the device and/or system. In some aspects, the device can further comprise an air pump and hose together configured to deliver lightly pressurized air to the device. In some aspects, the device can be configured to attach to an existing air pump and hose. In some aspects, the patient specific mandibular component and/or patient specific nasal component can be removable and/or replaceable.

In some aspects, the device can be configured to deliver pressurized air to a subject's airway to keep the subject's upper airway open to thereby reduce and/or prevent sleep apnea. In some aspects, the device can be configured to be used with an adult subject and/or an adolescent subject.

In some embodiments, provided herein are methods of treating and/or ameliorating sleep apnea, hypopnea syndrome, and/or obstructive sleep apnea. Such methods can comprise providing a subject suffering from and/or susceptible to sleep apnea, hypopnea syndrome, and/or obstructive sleep apnea, providing a CPAP device and/or system as disclosed herein, and using the CPAP device and/or system on the subject to treat and/or ameliorate sleep apnea, hypopnea syndrome, and/or obstructive sleep apnea in the subject. In some aspects, the CPAP device and/or system can be used on the subject while the subject is sleeping. In some aspects, the subject can be an adult human subject or an adolescent human subject.

Accordingly, it is an object of the presently disclosed subject matter to provide improved devices and systems configured to deliver pressurized air to an oral and/or nasal passageway of a subject to treat sleep apnea and related conditions. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 is a perspective view from one side of one embodiment of a CPAP device as disclosed herein;

FIG. 2 is an exploded view of the CPAP device of FIG. 1;

FIG. 3 is an end perspective view of one embodiment of a CPAP device as disclosed herein;

FIG. 4 is an orthogonal view from one of one embodiment of a CPAP device as disclosed herein;

FIG. 5 is a perspective view from one side of another embodiment of a CPAP device as disclosed herein;

FIG. 6 is a perspective view of one embodiment of a CPAP system as disclosed herein; and

FIGS. 7A through 7D are profile (FIG. 7A) and cross-sectional (FIGS. 7B-7D) schematic images depicting a valve assembly in an embodiment of a CPAP system as disclosed herein.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “an opening” includes a plurality of such openings, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

In some embodiments provided herein are CPAP devices and/or systems comprising, among other things, a custom mandibular appliance, derived from an impression of subject's/patient's teeth, with oral air delivery built in, and a custom nasal PAP that is derived from an impression of the subject's/patient's nose (configured in some aspects as a custom cannula). Such devices and/or systems can be configured such that no straps of any kind are required to retain the device in place on a subject/patient while in use. Moreover, such devices and/or systems can be designed for both mouth and nose breathers, as simultaneous pressure can be delivered to one or both airways as needed. Still yet, changes to a subject's/patient's face due to age, weight gain/loss, etc., may in some embodiments not require a new device since the device can easily be adjusted. Given the design and configuration of the disclosed devices and systems changes in the shape of a subject's/patient's face are less impactful since there is no reliance on a mask to create a seal as in traditional CPAP devices.

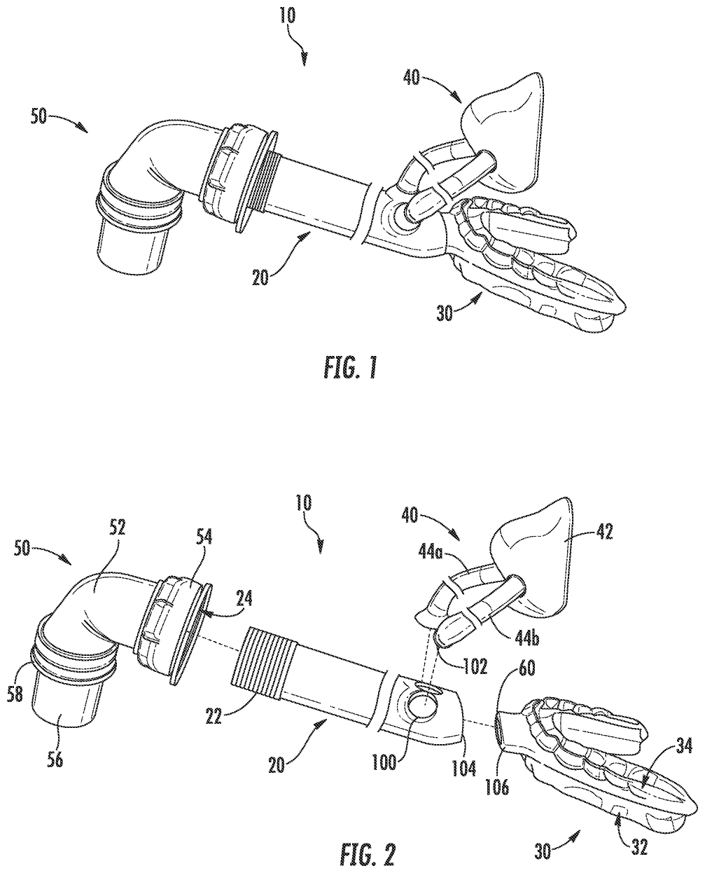

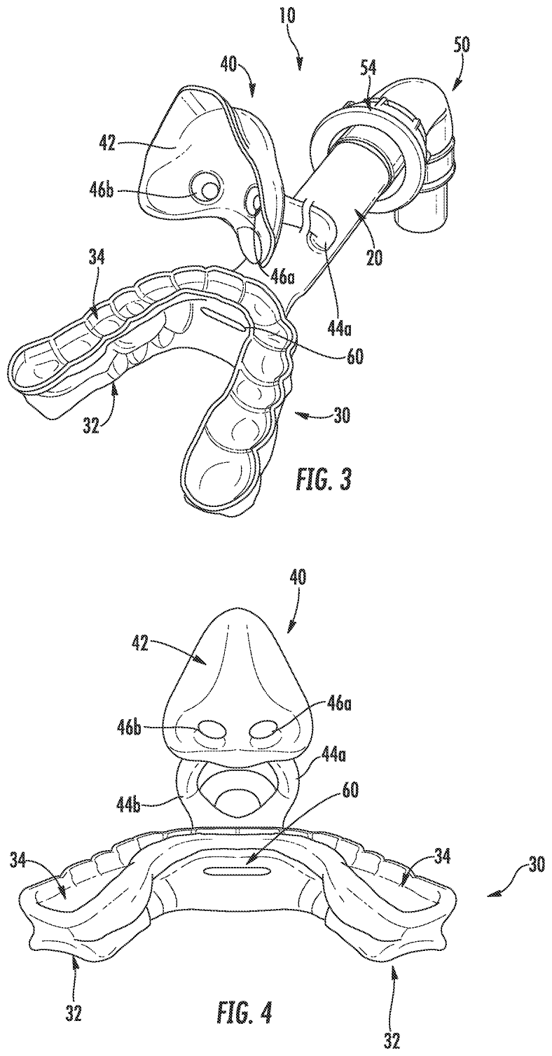

As illustrated in FIG. 1-5, the disclosed CPAP devices 10 and/or systems can comprise a mandibular component 30 and a nasal component 40, both stemming from, or fluidly connected to, a main body 20. Main body 20 can be configured to connect to an air supply such as for example a hose 84 from an air pump 82 (see FIG. 6). In some embodiments, air pumped into main body 20 can pass through or flow into nasal component 40 and/or mandibular component 30, either separately or simultaneously. As such, when worn by a patient or subject, continuous positive air pressure can be provided to the patient or subject via the nose and/or mouth.

Noticeably absent from the device depicted in FIGS. 1-5 is a strap(s) or other structure to secure the device to a patient's/subject's face and head. Unlike traditional CPAP devices there is no mask covering the nose and mouth, and no requirement to create a seal between the face and the edges of the mask. Thus, no straps or other securing mechanisms or means are needed. Instead, oral retention via mandibular component 30 is used to hold CPAP device 10, including mandibular 30 and nasal 40 components, in place while being used by a patient. A patient using a device as disclosed herein can retain the device in place by biting down on mandibular component 30. The user's or subject's mouth, and particularly lips, can in some embodiments create a seal around the oral air passageway in mandibular component 30, while nasal component 40 fits securely against the nose and nostrils of the user or subject to create an air-tight seal, or at least substantially air-tight seal, for the nasal air passageway. Such a configuration is designed for subjects that are mouth breathers as well as subjects that are nose breathers, as simultaneous air pressure can, in some embodiments, be delivered to one or both airways, as needed.

The mask-less and/or strap-less design of the disclosed devices and systems is configured to improve patient tolerance and compliance by substantially reducing or minimizing uncomfortable and intrusive straps and headgear, particularly while sleeping. By eliminating the mask of traditional CPAP devices there is less concern with improper fit and leaking caused by movement during sleep. This less intrusive system, as illustrated in FIGS. 1 through 6, is configured to maintain proper placement and air-tight seals by virtue of the oral retention that is less likely to be affected by movement during sleep. The customizable nature of the disclosed devices and systems, and particularly the mandibular and nasal components, contributes to the improved fit and usability as compared to existing CPAP devices.

Continuing with FIGS. 1 to 6, main body 20 can comprise a substantially tubular housing connecting hose coupler 50 with the nasal 40 and mandibular 30 components. Thus, main body 20 can in some embodiments act as a conduit for delivering pressurized air to nasal 40 and mandibular 30 components, and acting as a main structure from which these components extend. Main body 20 can be about 1 inch, about 2 inches, about 3 inches or about 4 inches long, and have a diameter of about 0.5 inches, about 1 inch or about 1.5 inches. Main body 20 can be made of any suitable material, including for example thermoplastics.

Turning now to FIG. 2, depicting the CPAP device 10 of FIG. 1, but in exploded view, hose coupler 50 can, in some embodiments, comprise a joint 52, collar 54, sleeve 56 and/or shoulder 58. Joint 52 can comprise a substantially tubular or hollow member acting as a conduit to transfer pressurized air from an air source to main body 20. Joint 52 can, in some embodiments, comprise a bend or angled portion altering the direction of flow or orientation of its respective ends to properly align collar 54 and sleeve 56. As depicted in FIG. 2, for example, joint 52 can comprise a 90°, or substantially 90°, bend from collar 54 to sleeve 56. However, in some embodiments, this bend can be less than about 90°, such as for example about 10°, about 20°, about 30°, about 40°, about 50°, about 60°, about 70°, or about 80°. In some embodiments there can be no bend or angle in joint 52 between collar 54 and sleeve 56. The orientation of couple 50 with collar 54, or main body 20 when attached to device 10, can be configured as desired and/or needed depending on the patient's needs or preference.

Hose coupler 50 can further comprise, at one end, collar 54, and at an opposing or second end, sleeve 56. Collar 54 can, in some embodiments, comprise a threaded coupling member configured to threadingly engage the terminal end of main body 20. To elaborate, in some aspect main body 20 can comprise a threaded male end 22 configured to threadingly engage a threaded female end 24 of collar 54 to thereby provide a secure connection between hose coupler 50 and main body 20. In some embodiments, collar 54 can be rotatably attached to coupler 50 such that collar 54 can be freely turned or rotated to engage threaded male end 22 of main body 20. As would be appreciated by one of ordinary skill in the art, any other suitable connection mechanism or system, e.g. slide and twist, snap-fit, compression fit, etc., between hose coupler 50 and main body 20 can be provided without departing from the scope of the instant disclosure.

Continuing with FIG. 2, hose coupler 50 can further comprise a hose connector, or sleeve 56, configured to attach to or otherwise receive a hose or conduit, e.g. an air hose (see, e.g., hose 84 in FIG. 6). By way of example and not limitation, sleeve 56 can be of a diameter sufficient to engage a hose from an air source, such as for example by having an outer diameter substantially equal to or slightly less than an inner diameter of an air hose such that the air hose can slidingly but securely engage sleeve 56. In some embodiments, a shoulder 58 can be positioned at an upper portion of sleeve 56, against which an air hose can abut when slidingly engaging sleeve 56. In some embodiments, a hose clamp or other securing mechanism can be provided to secure an air hose to sleeve 56.

Continuing with FIG. 2, mandibular component 30 can comprise upper 34 and lower 32 portions. Mandibular component 30 can, in some embodiments, be described as a mouth guard or mouth piece configured to conform to the upper and lower teeth of a subject or patient. Mandibular component 30 can be custom made for a particular patient by forming the component, including the upper 34 and lower 32 portions, from a virtual impression (digital impression) or manual impression (literal impression) of the patient's teeth. A virtual impression can be obtained by any suitable approach, including for example a CT scan, and/or computer-aided design (CAD), whereby the virtual data can be used to create a thermoplastic (e.g. BIOCRYL®, Scheu Dental, GmbH, Iserlohn, Germany) or biocompatible clear material (E-Guard, EnvisionTEC, Inc., Dearborn, Mich., United States of America) version by injection molding, pour molding, 3D printing, etc. Obtained and/or generated virtual data can be optimized and/or formatted in any suitable file type, including for example STEP files, STL files, IGES, and more, for use in formation of the component, for example by 3D printing. A manual impression can be obtained from a patient using existing dental and orthodontic practices to create a dental impression, whereby a thermoplastic (e.g. BIOCRYL®, Scheu Dental, GmbH, Iserlohn, Germany) or biocompatible clear material (E-Guard, EnvisionTEC, Inc., Dearborn, Mich., United States of America) form can version can be created via injection molding, pour molding, 3D printing, etc.

Mandibular component 30 can be affixed to and extend from main body 20, and can in some embodiments include an oral air passage 60, as depicted in FIGS. 2, 3 and 4. Oral air passage 60 can be configured to allow pressurized air from main body 20 to pass into the mouth of the patient when the device is in use, i.e. mandibular component 30 is inside the patient's mouth. Oral air passage 60 can, in some embodiments, be configured as a substantially horizontal slot or narrow opening to thereby allow sufficient air to pass through without significantly impeding the patient's bite or natural resting place of the upper and lower jaws when aligned with mandibular component 30.

As depicted in the figures, particularly FIGS. 2, 3 and 4, nasal component 40 comprises a cupped portion 42 configured to conform to a patient's nose, with two nasal air passages 46a, 46b configured to align with a patient's nostrils. Nasal component 40 extends from and is in fluid communication with main housing 20 via cannulae 44a and 44b, as depicted in FIGS. 2, 3 and 4. The one or more cannulae 44a and 44b act as structural supports to properly position nasal component 40 on the nose when the device is retained by a subject via oral retention, and also provide a conduit through which pressurized air can pass from main housing 20 and into one or both nostrils of the nose via nasal air passages 46a, 46b. Like mandibular component 30, nasal component 40 comprises a custom nasal device, or nasal PAP, that can be derived from an impression of the subject's/patient's nose. Since it is configured to the exact, or substantially exact, dimensions of the patient's nose nasal component 40 can in some embodiments act as a custom cannula in that an air-tight seal, or substantially air-tight seal, is created on the nose that allows pressurized air to pass into the nostrils.

Nasal component 40 can be custom made for a particular patient by forming the component, including the cupped portion 42, from a virtual impression (digital impression) or manual impression (literal impression) of the patient's nose. A virtual impression can be obtained by any suitable approach, including for example a CT scan, whereby the virtual data can be used to create a thermoplastic (e.g. BIOCRYL®, Scheu Dental, GmbH, Iserlohn, Germany) or biocompatible clear material (E-Guard, EnvisionTEC, Inc., Dearborn, Mich., United States of America) version by injection molding, pour molding, 3D printing, etc. Obtained and/or generated virtual data can be optimized and/or formatted in any suitable file type, including for example STEP files, STL files, IGES, and more, for use in formation of the component, for example by 3D printing. A manual impression can be obtained from a patient using existing practices to create an impression, whereby a thermoplastic (e.g. BIOCRYL®, Scheu Dental, GmbH, Iserlohn, Germany) or biocompatible clear material (E-Guard, EnvisionTEC, Inc., Dearborn, Mich., United States of America) form can version can be created via injection molding, pour molding, 3D printing, etc.

In some embodiments, the disclosed CPAP devices can be provided in a kit. In some embodiments, such a kit can comprise main body 20 and/or hose coupler 50, along with mandibular 30 and nasal 40 components already customized to the patient, or ready to be customized to the patient. For example, a kit can comprise mandibular 30 and nasal 40 component blanks or templates, or instructions for obtaining virtual data for the same, such that these components can be customized to the patient. Once customized to the patient the mandibular 30 and nasal 40 components can be assembled with the main body 20 and/or hose coupler 50 to complete the CPAP device. Such kits can further comprise instructions, materials and tools as needed.

The mandibular 30 and nasal 40 components form oral and nasal seals, respectively, thereby allowing the disclosed devices and systems to provide dual delivery of pressurized air to both the nose and mouth. Notably, this dual delivery is provided without the use of a full face mask as is required by current devices.

Together the mandibular component 40 and nasal component 40 provide a mask-less or strap-less CPAP device that is a full phase system. That is, the full phase system provides dual delivery, i.e. mouth and nose, of continuous positive air pressure. Such a configuration allows all patients, both mouth breathers and nose breathers, to use the device, without the discomfort and unwanted effects of a full mask design currently available.

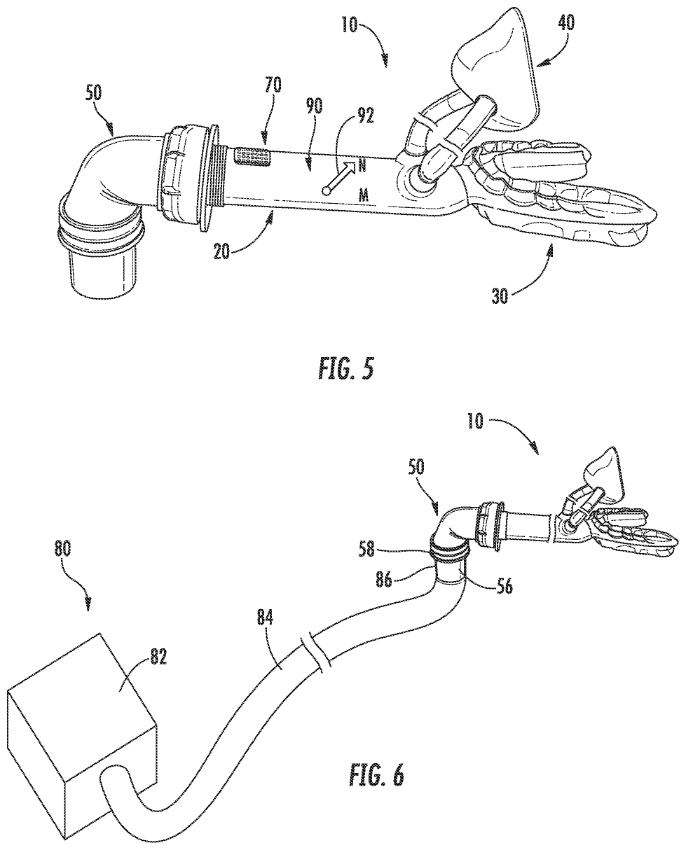

FIG. 5 illustrates another embodiment of a CPAP device 10 comprising an optional CO₂ exchanger 70, or other ventilating component, configured to allow expired air, or CO₂, from the patient/subject to exit device 10, and particularly main body 20. By way of example and not limitation, CO₂ exchanger 70 can be positioned on a surface of main body 20 between hose coupler 50 and nasal component 40, as depicted in FIG. 5. Alternatively, CO₂ exchanger 70 can, in some embodiments, be positioned on the main body between nasal component 40 and mandibular component 30, or on a surface of the main body between the cannulae 44a/44b.

FIG. 5 also illustrates an embodiment of a CPAP device 10 comprising a directional valve system 90 configured to direct, divert or otherwise control air flow to within the interior of main body 20 such that a user can control air flow to the mouth M, via mandibular component 30, the nose N, via nasal component 40, or both. In some embodiments, directional valve system 90 can comprise a selector component 92 on an exterior of main body 20, wherein the selector component 92 can be moved or positioned by a user to control an internal diverter or valve to control air flow to the mouth M, the nose N, or both.

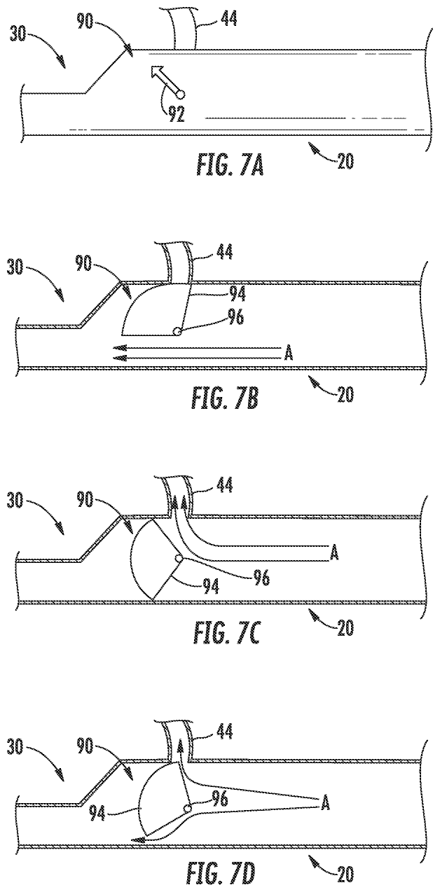

FIGS. 7A through 7D illustrate further details of directional valve system 90. FIG. 7A is a close-up view of main body 20 and directional valve system 90, including selector component 92. FIGS. 7B through 7D are cross-sectional views of FIG. 7A, and depicting further details of directional valve system 90. As shown in FIGS. 7B through 7D, directional valve system 90 can, in some embodiments, comprise diverter 94 rotatable about an axis 96. In some aspects, diverter 94 is rotatable around axis 96 such that diverter 94 can be positioned in any one of a plurality of positions, diverter 94 can be positioned in any one of a plurality of positions, including one of three positions illustrated by FIGS. 7B, 7C and 7D. In a first position shown in FIG. 7B, diverter 94 can be positioned to completely or substantially block air flow A to cannulae 44 (directed to nasal component 40) and allow all or substantially all air flow A to mandibular component 30. In a second position shown in FIG. 7C, diverter 94 can be positioned to completely or substantially block air flow A to mandibular component 30 and allow all or substantially all air flow A nasal component 40 by way of cannulae 44. In a third position, as shown in FIG. 7D, diverter 94 can be positioned to allow air flow A to both mandibular component 30 and nasal component 40 by way of cannulae 44. Moreover, in some embodiments, directional valve system 90 can be tuned or positioned to allow precise control over proportional air flow A to one or both mandibular component 30 and/or nasal component 40 (e.g. about 50% to the nose and about 50% to the mouth, or about 25% to the nose an about 75% to the mouth, and vice versa). Such a configuration can allow for a patient or subject to alter and control air flow to one or both airways to optimize the effectiveness and/or comfort of the CPAP devices and systems disclosed herein.

The disclosed devices and systems can be configured to be used with standard or existing CPAP pumps, hoses and accessories, as shown in FIG. 6. That is, the disclosed mask-less and/or strap-less CPAP device can be configured to be used with an existing CPAP air pump and corresponding hose to deliver a desired and/or prescribed amount of pressurized air to a patient via the nose and/or mouth. In some embodiments a patient with an existing CPAP unit or system that is configured to deliver lightly pressurized air through a hose that is hooked to a mask can detach the current mask from the hose and attached the disclosed device in place thereof.

Alternatively, in some embodiments the disclosed device, i.e. mask-less and strap-less CPAP device, can be provided with an air pump and flexible hose/tubing together configured to deliver lightly pressurized air to the device. In some embodiments these components, including for example a pump, hose and mask-less/strap-less CPAP device, can be provided as a complete CPAP system, unit or kit. In some embodiments instructions for use can also be provided.

As depicted in FIG. 6, the CPAP device 10, and particularly main body 20, can be connected to an air source 80, including for example an air pump 82 or CPAP pump, by a hose 84 or similar component. Such a hose 84 can include for example a flexible hose connected to an air pump 82, such as a standard CPAP air pump. Hose coupler 50, and particularly sleeve 56, can in some embodiments be configured to connect to a standard sized air hose commonly provided with an air pump for CPAP systems.

Use of the disclosed device, in conjunction with an air supply, i.e. a pump, can act like a splint to keep the subject's upper airway open to thereby reduce and/or prevent sleep apnea. The disclosed devices are configured as described herein to deliver a desired and/or prescribed amount of pressurized air to a patient via the nose and/or mouth on demand, i.e. as needed depending on how the patient/subject breathes. Moreover, in some embodiments, the disclosed CPAP devices and systems are configured to provide a reduced amount of pressurized air while still achieving the same or substantially similar results and efficacies in treading sleep apnea and related conditions. By way of example and not limitation, the disclosed CPAP devices and systems can be as effective as existing CPAP devices while delivering about 25% less pressure, or about 10% to about 50%, or about 20% to about 40% less pressure. For example, whereas most existing CPAP devices provide pressurized air at about 8 cm water (cm H₂O), the disclosed CPAP devices can provide pressurized air at about 6 cm water, an about 25% reduction.

In some embodiments, the disclosed CPAP devices, such as depicted in FIGS. 1 3, 4 and 5, for example, can be formed, molded or 3D printed as as a complete system, and/or single unitary device. Alternatively, in some embodiments, one or more components of the disclosed devices can be replaceable or otherwise configured to be changed out for a new component as needed. By way of example and not limitation, the mandibular component and/or nasal component can be configured to be detached and replaced with new and/or updated components. For example, referring to FIG. 2, mandibular component 30 can be configured with end 106 adapted to attach to or insert into receiving portion 104 of main body 20. Moreover, again referring to FIG. 2, nasal component 40 can be configured with ends 102 (each at a terminal end of each of cannulae 44a and 44b) adapted to attach to or insert into receiving ports 100 of main body 20. Any suitable attachment mechanism, e.g. threads, snap-fit, twist and lock, etc. can be used to join these elements. In addition, hose coupler 50 can be configured to be changed out or replaced as needed. Such a configuration can allow for worn or outdated components to be changed as needed without having to replace the entire device.

In some embodiments the disclosed CPAP devices and/or systems can be configured to be used with adults or children. Since the nasal component and mandibular component are specific to the patient, a suitable fit can be achieved no matter the age, weight, size, etc. of the patient. Unlike existing CPAP devices that are poorly configured to be used in pediatrics, the disclosed CPAP devices and/or systems are ideally suited for pediatric use. The custom fit components allow an air-tight seal to be formed at both the nasal component and mandibular component even in small children. Moreover, the absence of straps and masks make the disclosed devices less obtrusive and therefore more accepted by children. In some embodiments the disclosed CPAP devices and/or systems can be colored or provided with designs/motifs that are appealing to children.

In some embodiments, provided herein are methods of treating and/or ameliorating sleep apnea, hypopnea syndrome, and/or obstructive sleep apnea. Such methods can comprise providing a subject suffering from and/or susceptible to sleep apnea, hypopnea syndrome, and/or obstructive sleep apnea, providing a CPAP device and/or system as disclosed herein, and using the CPAP device and/or system on the subject to treat and/or ameliorate sleep apnea, hypopnea syndrome, and/or obstructive sleep apnea in the subject. In some aspects, the CPAP device and/or system can be used on the subject while the subject is sleeping. In some aspects, the subject can be an adult human subject or an adolescent human subject.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A continuous positive airway pressure (CPAP) device and/or system comprising:
a mandibular component;
a nasal component;
a main body connected to and in fluid communication with the mandibular component and the nasal component; and
a hose coupler configured to receive a hose providing pressurized air from an air source,
wherein the main body is configured to deliver the pressurized air from the air hose to one or both of the mandibular component and the nasal component, wherein the mandibular component is configured to deliver pressurized air to an oral passageway of a subject, wherein the nasal component is configured to deliver pressurized air to a nasal passageway of a subject.

2. The CPAP device and/or system of claim 1, wherein the mandibular component comprises an impression of a subject's teeth.

3. The CPAP device and/or system of any of claims 1 to 2, wherein the mandibular component comprises an upper portion and lower portion configured to conform to the upper and lower teeth, respectively, of a subject.

4. The CPAP device and/or system of any of claims 1 to 3, wherein the device and/or system is configured to be retained on a subject's face by oral retention on the mandibular component.

5. The CPAP device and/or system of any of claims 1 to 4, wherein the mandibular component comprises an oral air passage configured to deliver pressurized air to an oral passageway of a subject.

6. The CPAP device and/or system of any of claims 1 to 5, wherein the nasal component comprises an impression of a subject's nose.

7. The CPAP device and/or system of any of claims 1 to 6, wherein the nasal component comprises one or more cannulae configured to position the nasal component on a subject's nose, and configured to deliver pressurized air from the main body to the subject's nose.

8. The CPAP device and/or system of any of claims 1 to 7, wherein the nasal component further comprises two nasal air passages configured to align with a subject's nostrils.

9. The CPAP device and/or system of any of claims 1 to 8, wherein the mandibular component is configured to be positioned in a subject's mouth to create a substantially air-tight seal for the oral passageway, wherein the nasal component is configured to be positioned on a subject's nose to create a substantially air-tight seal for the nasal passageway.

10. The CPAP device and/or system of any of claims 1 to 9, wherein the device and/or system is configured to provide dual delivery of pressurized air to both the nose and mouth.

11. The CPAP device and/or system of any of claims 1 to 10, wherein the device and/or system is devoid of retaining straps and a mask.

12. The CPAP device and/or system of any of claims 1 to 11, wherein the device and/or system further comprises a CO₂ exchanger configured to allow expired air to exit the device and/or system.

13. The CPAP device and/or system of any of claims 1 to 12, further comprising an air pump and hose together configured to deliver lightly pressurized air to the device and/or system.

14. The CPAP device and/or system of any of claims 1 to 13, wherein the device and/or system is configured to attach to an existing air pump and hose.

15. The CPAP device and/or system of any of claims 1 to 14, wherein the mandibular component and/or nasal component are removable and/or replaceable.

16. The CPAP device and/or system of any of claims 1 to 15, further comprising a diverter component configured to control a flow of air to the mandibular component, the nasal component, or both.

17. The CPAP device and/or system of any of claims 1 to 16, wherein the device and/or system is configured to deliver pressurized air to a subject's airway to keep the subject's upper airway open to thereby reduce and/or prevent sleep apnea.

18. The CPAP device and/or system of any of claims 1 to 17, wherein the device and/or system is configured to be used with an adult subject and/or an adolescent subject.

19. A mask-less and/or strap-less CPAP device configured to provide dual delivery of continuous pressurized air to a mouth and nose of a subject, the device comprising a patient specific mandibular component and patient specific nasal component, wherein the device is configured to be retained in place by oral retention on the mandibular component.

20. The mask-less and/or strap-less CPAP device of claim 19, further comprising:
a main body connected to and in fluid communication with the patient specific mandibular component and the patient specific nasal component; and
a hose coupler configured to receive a hose providing pressurized air from an air source,
wherein the main body is configured to deliver the pressurized air from the air hose to one or both of the patient specific mandibular component and the patient specific nasal component, wherein the patient specific mandibular component is configured to deliver pressurized air to an oral passageway of a subject, wherein the patient specific nasal component is configured to deliver pressurized air to a nasal passageway of a subject.

21. The mask-less and/or strap-less CPAP device of any of claims 19 to 20, wherein the device is configured to be used by a subject that is a mouth breather, nose breather, or both.

22. The mask-less and/or strap-less CPAP device of any of claims 19 to 21, wherein the patient specific mandibular component comprises an impression of a subject's teeth.

23. The mask-less and/or strap-less CPAP device of any of claims 19 to 22, wherein the patient specific mandibular component comprises an upper portion and lower portion configured to conform to the upper and lower teeth, respectively, of a subject.

24. The mask-less and/or strap-less CPAP device of any of claims 19 to 23, wherein the device is configured to be retained on a subject's face by oral retention on the patient specific mandibular component.

25. The mask-less and/or strap-less CPAP device of any of claims 19 to 24, wherein the patient specific mandibular component comprises an oral air passage configured to deliver pressurized air to an oral passageway of a subject.

26. The mask-less and/or strap-less CPAP device of any of claims 19 to 25, wherein the patient specific nasal component comprises an impression of a subject's nose.

27. The mask-less and/or strap-less CPAP device of any of claims 19 to 26, wherein the patient specific nasal component comprises one or more cannulae configured to position the patient specific nasal component on a subject's nose, and configured to deliver pressurized air from the main body to the subject's nose.

28. The mask-less and/or strap-less CPAP device of any of claims 19 to 27, wherein the patient specific nasal component further comprises two nasal air passages configured to align with a subject's nostrils.

29. The mask-less and/or strap-less CPAP device of any of claims 19 to 28, wherein the patient specific mandibular component is configured to be positioned in a subject's mouth to create a substantially air-tight seal for the oral passageway, wherein the patient specific nasal component is configured to be positioned on a subject's nose to create a substantially air-tight seal for the nasal passageway.

30. The mask-less and/or strap-less CPAP device of any of claims 19 to 29, wherein the device is configured to provide dual delivery of pressurized air to both the nose and mouth.

31. The mask-less and/or strap-less CPAP device of any of claims 19 to 30, wherein the device is devoid of retaining straps and a mask.

32. The mask-less and/or strap-less CPAP device of any of claims 19 to 31, wherein the device further comprises a CO₂ exchanger configured to allow expired air to exit the device and/or system.

33. The mask-less and/or strap-less CPAP device of any of claims 19 to 32, further comprising a diverter component configured to control a flow of air to the mandibular component, the nasal component, or both.

34. The mask-less and/or strap-less CPAP device of any of claims 19 to 33, further comprising an air pump and hose together configured to deliver lightly pressurized air to the device.

35. The mask-less and/or strap-less CPAP device of any of claims 19 to 34, wherein the device is configured to attach to an existing air pump and hose.

36. The mask-less and/or strap-less CPAP device of any of claims 19 to 35, wherein the patient specific mandibular component and/or patient specific nasal component are removable and/or replaceable.

37. The mask-less and/or strap-less CPAP device of any of claims 19 to 36, wherein the device is configured to deliver pressurized air to a subject's airway to keep the subject's upper airway open to thereby reduce and/or prevent sleep apnea.

38. The mask-less and/or strap-less CPAP device of any of claims 19 to 37, wherein the device is configured to be used with an adult subject and/or an adolescent subject.

39. A method of treating and/or ameliorating sleep apnea, hypopnea syndrome, and/or obstructive sleep apnea, the method comprising:
providing a subject suffering from and/or susceptible to sleep apnea, hypopnea syndrome, and/or obstructive sleep apnea;
providing a CPAP device and/or system of any of claims 1 to 38;
using the CPAP device and/or system on the subject to treat and/or ameliorate sleep apnea, hypopnea syndrome, and/or obstructive sleep apnea in the subject.

40. The method of claim 39, wherein the CPAP device and/or system is used on the subject while the subject is sleeping.

41. The method of any of claims 39 to 40, wherein the subject is an adult human subject or an adolescent human subject.
